(19) United States
(12) Patent Application Publication
(10) Pub. No.: US 2015/0223683 A1
(43) Pub. Date: Aug. 13, 2015

(54) System For Synchronously Sampled Binocular Video-Oculography Using A Single Head-Mounted Camera

(71) Applicants: Natan Simcha Davidovics, Baltimore, MD (US); Mehdi Rahman, Baltimore, MD (US); Nicolas Sebastian Valentin Contreras, Baltimore, MD (US); Charles Coleman Della Santina, Towson, MD (US)

(72) Inventors: Natan Simcha Davidovics, Baltimore, MD (US); Mehdi Rahman, Baltimore, MD (US); Nicolas Sebastian Valentin Contreras, Baltimore, MD (US); Charles Coleman Della Santina, Towson, MD (US)

(73) Assignee: LABYRINTH DEVICES, LLC, Towson, MD (US)

(21) Appl. No.: 14/613,993

(22) Filed: Feb. 4, 2015

Related U.S. Application Data
(60) Provisional application No. 61/938,134, filed on Feb. 10, 2014.

(51) Int. Cl. A61B 3/113; A61B 3/02; A61B 3/15; A61B 3/14; A61B 3/00

(52) U.S. Cl. CPC A61B 3/113; A61B 3/145; A61B 3/0025; A61B 3/152; A61B 3/0091; A61B 3/02; G02B 5/18

(57) ABSTRACT

A one-camera, binocular, video-oculography (1CBVOG) system for measuring the movement of both of the eyes of a test subject, while the head of the test subject is undergoing a period of vestibular or oculomotor stimulation, includes: (a) a base frame, (b) a binocular imaging component, including a video camera adapted to capture a sequence of images containing both of the eyes of the test subject, (c) an optical component, (d) an illumination source, (e) a sensor module that senses translational and rotational motion of the head along and about three, mutually orthogonal axes that approximately align with the axes of the inner ears' semicircular canals, and (f) a computing device configured to quantify and measure the movement of the test subject's eyes from the sequence of captured images.

### CROSS-REFERENCE TO RELATED APPLICATION

This application claims the benefit of Provisional Patent Application No. 61/938,134, filed Feb. 10, 2014 by the present inventors. The teachings of this earlier application are incorporated herein by reference to the extent that they do not conflict with the teaching herein.

### BACKGROUND OF THE INVENTION

1. Field of the Invention

This invention relates to binocular video-oculography (BVOG, synchronous recordings of the angular position and movement of both eyes in a test subject). More particularly, the present invention relates to inexpensive, real-time, three-dimensional, simultaneously-sampled, binocular video-oculography with a single camera.

2. Description of Prior Art

Efficiently and accurately evaluating vestibular function in individuals with spontaneous or post-traumatic vertigo and disequilibrium is a major challenge for clinicians in both military and civilian settings. While a careful history and physical exam can often distinguish peripheral vestibular disorders due to inner ear dysfunction from central disorders that may be life-threatening and require more intensive care, dizzy patients are frequently misdiagnosed and inappropriately treated despite undergoing expensive tests. A recent cost-utility analysis of emergency department care by Newman-Toker and colleagues revealed that although hundreds of millions of dollars are spent on imaging each year in the U.S. in attempts to rule out central nervous system causes of vertigo, one third of strokes that cause vertigo are missed initially. Both in civilian settings and in military field hospitals, this problem is compounded by a relative lack of clinician training with regard to efficient diagnosis of inner ear disorders. As a result, many of the over 4 million emergency department and acute care visits for vertigo or dizziness in the US each year, which in aggregate cost over $4 billion annually, result in misdiagnosis. Patients with inner ear conditions such as vestibular neuritis (or labyrinthitis) and benign paroxysmal positional vertigo (BPPV) are often imaged and admitted unnecessarily instead of being treated and discharged. In contrast, patients with dangerous brainstem or cerebellar strokes may be sent home without appropriate treatment. Accurately identifying and treating the ˜25% of vertigo cases that are due to peripheral (i.e., inner ear) vestibular disorders could save hundreds of millions of dollars per year while achieving better health outcomes and reducing long-term disability.

Like other causes of vertigo, mild traumatic brain injury (mTBI) due to closed head trauma and concussive injury is a common, vexing and costly diagnostic challenge. With over 8 million individuals suffering head injuries in the United States annually, over 400 K/yr of them hospitalized, 80% of those meeting criteria for mTBI, and the majority of that subset complaining of nonspecific dizziness, disequilibrium and disorientation that can mimic a peripheral vestibular disorder, the need for accurate quantification of vestibular function after head trauma is large and largely unmet by currently available technologies in military theater and in community hospital settings.

Reflecting this concern, both the US Department of Defense and the National Institutes of Health have identified efficient differentiation between acute central and peripheral vestibular disorders as a high priority public health goal.

A majority of otolaryngologists, neurologists and emergency physicians consider diagnosis and treatment of vestibular disorders to be confusing and frustrating. In part, this situation stems from continued reliance on old and nonspecific diagnostic technologies. Caloric electronystagmography (ENG), in which warm and cool water irrigation of each ear canal is used to excite or inhibit the horizontal semicircular canal and elicit eye movements via the vestibulo-ocular reflex (VOR), has been considered a “gold standard” vestibular test since it was pioneered a century ago (resulting in a 1914 Nobel Prize). However, caloric ENG has significant disadvantages: (1) it only tests the function of the two horizontal semicircular canals and tells nothing about the four other canals; (2) it probes inner ear function with a −0.01 Hz stimulus that is almost completely irrelevant to the normal physiology of the labyrinth and VOR, which mainly evolved to stabilize the eyes during quick, high-acceleration head movements with most spectral energy around 0.1-40 Hz; (3) it is time-consuming (a typical ENG appointment takes 60 minutes); (4) it is unpleasant for the patient, due to the vertigo and nausea it elicits; (5) its eye movement measurement technique (electro-oculography, EOG) is imprecise, essentially limited to 1-dimensional (1D) horizontal movements and subject to noise and drift that mandate use of filters that limit temporal resolution; and (6) it tells nothing about the status of the utricle and saccule.

Over the past 100 years, rotary chair testing became popular as a means to overcome some of these problems. During a rotary chair test, a patient sits atop a rotating motor while eye movements driven by the VOR are monitored either using EOG or using video cameras. Because of the high torques required to move an entire human body, rotary chairs are expensive and large, requiring such a large commitment of capital (˜$250 K), floor space (−100 sf) and dedicated staff (˜$30-40 K/year for salary and fringe) that very few physician offices and only a small minority of hospitals have installed one. The vast majority of rotary chairs in current clinical use have insufficient torque to move the whole body at more than ˜1 Hz, so they fail to probe VOR function in the higher frequency range most relevant to normal VOR function and are incapable of identifying which labyrinth is abnormal in patients with mild unilateral deficits such as might occur in mTBI. Moreover, like caloric ENG, rotary chairs typically only test horizontal canal function, because they are limited to rotating about an Earth-vertical axis.

Over the past 50 years, the magnetic scleral coil technique has become the gold standard for measurement of eye movements, because it allows measurement of 3D rotational eye position at high sample rates. However, although it has long been used in research laboratories, it is impractical for routine clinical use, because it is uncomfortable for patients and requires expensive equipment and highly skilled examiners.

Over the past 25 years, the field has moved steadily away from low velocity, low acceleration, low frequency caloric and rotary chair stimuli and toward use of is quick, high-acceleration head movements that probe VOR function in the frequency range for which the reflex normally dominates eye stabilization. Responses to quick “head impulse” movements mainly depend on the semicircular canal most excited by the stimulus (while that canal's coplanar mate in the opposite ear is inhibited until effectively silent), so the “head impulse test” (HIT, also called the “head thrust test”) can test the left and right ears with much greater specificity than a typical rotary chair. Moreover, by grasping a patient's head and moving it briskly around the axes of the other two pairs of semicircular canals (i.e., the left-anterior and right-posterior [LA/RP] pair and the right-anterior and left posterior [RA/LP] pair), a clinician can selectively examine responses to stimulation of each of the six semicircular canals independently. Given these advantages, an extensive and growing body of literature has now made the head impulse test a de facto standard for examination of semicircular canal function.

One drawback of the HIT is that when performing it as a simple physical exam maneuver without a means of high-speed eye movement measurement, even highly experience clinicians are prone to missing the subtle eye movements that signify a vestibular deficit. This is especially problematic in patients with incomplete or long-standing deficits. Scleral coils have been used to overcome this drawback in research labs, but adoption of this complex and uncomfortable technique in routine clinical practice is unlikely.

Over the past 5-10 years, the availability of increasingly high-speed, high-resolution and lightweight cameras has revolutionized diagnostic testing of semicircular canal function. The “video head impulse test” (vHIT) is rapidly replacing caloric ENG and rotary chair testing as the preferred method of assessment in patients with possible vestibular disorders. At leading academic medical centers, caloric testing and rotary chairs are being replaced by a combination of vHIT and measurement of ocular and cervical vestibular-evoked myogenic potentials (oVEMPS and cVEMPS, which are used as objective measures of utricular and saccular function, respectively). Two products now dominate the market for vHIT systems, which have met with great enthusiasm by clinicians at conferences since being released over the past two years.

Despite the clear advantages of existing vHIT systems over the older technologies they are rapidly replacing, they too suffer from significant disadvantages. These systems—the ICS-Impulse sold by GN Otometrics and the EyeSeeCam system sold by InterAcoustics—are both optimized for high frame rate video acquisition of an eye's movement during yaw (horizontal) head rotations that are applied manually by an examiner grasping and quickly turning the patient's head. Like the systems upon which these products are based, other existing systems have multiple drawbacks including: (1) both are limited to 2D rotation measurements (horizontal and pitch). Neither can measure ocular torsion/counter-roll, so neither can accurately measure VOR responses to head rotation about the axes of any of the LA, RP, RA or RP semicircular canals; (2) both are limited to a single eye per camera. In their usual format, neither of these systems can measure binocular responses, which are especially important for characterizing otolith end organ function during translational head movements. The InterAcoustics system offers a dual camera version for a significantly higher price, but it requires two computers and two communication cables to run, and the resulting data are acquired asynchronously, making data analysis more noisy and error-prone because signals from the two separate cameras must be either synchronized via a triggered acquisition mode or interpolated in time to a common time base—a procedure that loses information, temporal resolution and accuracy; (3) neither system can be used to measure purely vestibular reflexes (i.e., responses to head rotation without the contribution of vision) unless the patient is in a completely darkened room or has an opaque bag drawn over his head. This makes test administration cumbersome and limits ability to situate the device in a clinic room that is not specifically dedicated and completely dark; (4) both systems have low spatial resolution, which translates to relatively high quantization noise in eye movement velocity data. At its maximum frame rate of 250 frame/s (fps), the ICS Impulse system's camera frame is 100×100 pixels. At EyeSeeCam's maximum rate of 600 fps, resolution is 160×60 pixels. Neither of these is sufficient to accurately measure ocular torsion, as required for measurement of 3D eye rotational position and movement; (5) neither system measures utricular or saccular function; and (6) neither system measures perception of subjective visual vertical. Kiderman et al. (U.S. Pat. No. 7,753,523 and U.S. Pat. No. 7,448,751) describes a goggle-based VOG system that includes a calibration laser and a digital camera connected to and powered by a laptop computer through a Firewire/IEEE1394-compliant connection. The digital camera may digitally center the pupil in both the X and Y directions. A calibration mechanism may be incorporated onto the goggle base. An electrooculography system may also be incorporated directly into the goggle. The VOG system may track and record 3D movement of the eye, track pupil dilation, head position and goggle slippage. An animated eye display provides data in an intuitive fashion. The VOG system is a modular design whereby the same goggle frame or base is used to build a variety of digital camera VOG systems. For example, using two digital cameras, each of which records the video images of one of a patient's eyes, can yield what we herein describe as a two-camera, binocular VOG (2CBVOG) system.

Brandt et al. (US 20060098087) and MacDougall, et al. (U.S. Pat. No. 7,731,360) describe VOG systems comprising a lightweight, head mounted frame and camera that also can only image a single eye. MacDougall, et al. developed nine such VOG systems at the University of Sydney from 1990-2003 and also summarized over 50 public presentations, publications and theses regarding this technology. See also MacDougall, et al. (U.S. Pat. No. 7,731,360), Furman et al. (WO2011002837 and US 20120133892) and Lewkowski (WO2007128034).

Bartomeu (U.S. Pat. No. 7,931,370) also discloses a helmet device incorporating two cameras to measure movements of a subject's two eyes—a 2CBVOG system. He additionally describes the use of a single camera in a smaller helmet intended for use in children with a smaller head and interpupillary distance. However, Bartomeu's single camera system, because it does not acquire images in which each eye's horizontal axis is parallel to either a row or column of the camera's image sensor, suffers from the significant disadvantage of requiring a computationally intensive and inherently information-losing mathematical reorientation of the image into the eye's coordinate system before reporting eye movement results. This significantly prolongs computational analysis time for each image, slowing the sample rate achieved with such a system.

Because each camera in these 2CBVOG systems requires its own communication interface to a controlling computer, such 2CBVOG systems typically require at least two communication cables, further increasing the mass and moment of inertia of such devices and incurring information transmission inefficiency because the host computer must frequently switch between two different data input streams while communicating with the two cameras. This information transmission inefficiency results in slower data acquisition rates and higher data acquisition latencies, consequently reducing ability to accurately measure the quick or high-acceleration eye movements that are especially important to investigators and clinicians seeking to discern the status of a patient or test subject's vestibular, neurologic and oculomotor function.

In addition to information transmission inefficiency, 2CBVOG systems suffer reduced performance due to asynchrony between the two cameras' shutters. Prior art approaches to mitigating this problem have included (1) triggered acquisition and (2) temporal interpolation of the two cameras' data streams on a common time base. Triggering acquisition using a master trigger to control the two cameras and enforce simultaneous closure of their shutters results in greater synchrony but comes at the cost of slower acquisition frame rates compared to the self-triggered, free-run mode in which the camera acquires images at its maximum possible rate. Temporal interpolation invariably incurs a loss of information due to the need to estimate rather than directly measure the eye position at a given moment in time.

Thus, there remains a need for a VOG system that can simultaneously and synchronously sample and compute the 3-dimensional, angular position and velocity of both eyes with a single head-mounted camera, while imaging each eye with its horizontal and vertical axes aligned to the rows and columns of the image sensor and while sensing translational and rotational motion of the head along and about three, mutually orthogonal axes that approximately align with the axes of the inner ears' semicircular canals. The present invention was developed to provide such a device. It is an especially useful device for improving the diagnosis of vestibular, oculomotor, neurologic, perceptual and attentional disorders.

### SUMMARY OF THE INVENTION

Recognizing the need for an improved VOG system that can simultaneously and synchronously sample the position and movement of both eyes with a single head-mounted camera while sensing translational and rotational motion of the head along and about three, mutually orthogonal axes that approximately align with the axes of the inner ears' semicircular canals, the present invention is generally directed to providing such a one-camera, binocular VOG (1CBVOG) system.

In a preferred embodiment, the present invention is a 1 CBVOG system for measuring the movement of both of the eyes of a test subject while the head of the test subject is undergoing a period of prescribed motion or alternative means of vestibular or oculomotor stimulation, such as head reorientation in a gravitational field, Valsalva or external ear pressure application maneuvers, optokinetic stimulation, presentation of visible targets, presentation of sound or head-tapping stimuli, caloric stimulation, electrical stimulation of the vestibular nerve, or other stimuli. This system includes: (a) a base frame adapted to be attached to the head of the test subject, (b) a binocular imaging component adapted to capture a sequence of images containing both of the eyes of the test subject during the period of vestibular or oculomotor stimulation, wherein this binocular imaging component includes a single video camera, (c) an optical component attached to the base frame and adapted to allow the binocular imaging component to capture a sequence of images containing both of the eyes of the test subject simultaneously and synchronously during the period of vestibular or oculomotor stimulation, wherein both of the eyes are imaged at the same effective moment in time and from effective vantage points that are within a prescribed number of degrees of the optic axis of each eye when that eye is in the center of its range of motion, (d) an illumination source, and (e) a sensor via which head motion data and signals from other sources can be acquired in synchrony with images from the imaging component, and (f) a computing device configured to communicate with the camera so as to quantify and measure the movement of both of the eyes of the subject from the sequence of captured images.

The system's optical component may further include: (c1) a beam splitting and redirecting mirror approximately centered on the test subject's midsagittal plane when the optical axis of the camera lies approximately within the midsagittal plane, and (c2) a plurality of alignment mirrors that are each aligned with the beam splitting and redirecting mirror and camera and wherein the alignment mirrors are configured so that the camera can simultaneously image both eyes without occluding the central region of either eye's visual field, and (c3) optionally, one or more of the following: a nonplanar mirror, a mirror having a gold reflective optical coating, a graded refractive index (GRIN) lens, and an optical conduit.

The system itself may also include: (f) at least one detachable optical filter shield configured to reversibly cover and occlude vision in one or both of the eyes of the test subject, wherein this shield includes an outer rim that encloses an optical filter and is attached to the base frame via magnetic or friction coupling, and with this optical filter chosen from the group including: (i) a long-pass optical filter configured to allow infrared light to pass while blocking light of any wavelength visible to humans, (ii) a band-pass optical filter that allows transmission of visible light over a narrow range centered on the peak emission wavelength chosen from the group of either a red or green or other color laser, and (iii) a stack of three optical filters, including a short-pass, a band-stop, and a long-pass filter, the combination of which results in a dual pass-band filter that allows transmission of infra-red light and also allows transmission of visible light over a narrow range centered on the peak emission wavelength chosen from the group of either a red or green or other color laser, (g) a motion or inertial sensor configured to sense translational movement, rotation movement and gravitational acceleration and having one, two or three axes of sensitivity and adapted to be immobilizably affixed to the head of the test subject and to output data signals that are synchronized to the camera images and act as measures of the orientation and movement of the head of the test subject, including head translation , head rotation and gravtational acceleration with respect to each of three mutually orthogonal axes when the inertial sensor is oriented so that its axes of sensitivity approximately align with the mean anatomic axes of the inner ear labyrinths' semicircular canals, (h) a diffraction grating affixed to a surface perpendicular to the test subject's naso-occipital axis, (i) a means for projecting a laser line, chosen from the group including visible spectrum lasers through the diffraction grating, and wherein the diffraction grating is configured so that the test subject can rotate the diffraction grating to adjust the orientation of the projected laser line until the test subject perceives the projected laser line as being vertical or horizontal.

The system's illumination source may include: (d1) a lamp and optical band-stop filter combination situated away from the test subject and emitting visible light to but excluding light at wavelengths within the visible light pass band of the detachable optical filter shields, and (d2) a light-emitting diode that emits visible light with sufficient intensity to cause a test subject's pupil to constrict to a pupil diameter smaller than that which occurs under infra-red lighting alone.

Furthermore, the system's computing device is programmed to quantify and measure the movement of both of the eyes of the test subject while accounting for and correcting for the possibility that the mean effective axis of eye rotation for horizontal (yaw) motion and the mean effective axis of eye rotation for vertical (pitch) components of eye rotation fail to intersect.

Additionally, the present invention can also be considered to be the method for utilizing a single-camera, binocular, video-oculographic (1CBVOG) system for measuring the movement of both of the eyes of a test subject while the head of said test subject is undergoing a period of prescribed vestibular or oculomotor stimulation, with these resulting measurements allowing a clinician who utilizes the system to improve his/her diagnosis of vestibular, oculomotor, neurologic, perceptual and attentional disorders in the test subject.

Thus, there has been summarized above (rather broadly and understanding that there are other preferred embodiments which have not been summarized above) the present invention in order that the detailed description that follows may be better understood and appreciated.

### BRIEF DESCRIPTION OF THE DRAWINGS

FIG. 1 is a quasi-frontal, perspective illustration of a system or apparatus for monitoring eye movement in a patient according to one embodiment of the present invention.

FIG. 2 is a quasi-side, perspective illustration of the embodiment of the present invention shown in FIG. 1 that further includes the computing device that is used to process the digital images coming from this system's camera that are used to quantify and measure the movement of both of the eyes of a test subject.

FIG. 3 is a frontal elevation view of the apparatus of FIG. 1, as viewed by an observer in front of and facing a test subject who would be wearing the apparatus.

FIG. 4 is a partial, right side view of the apparatus of FIG. 1, as viewed by an observer standing to the right of a test subject who would be wearing the apparatus.

FIG. 5 is an oblique elevation illustration of a right eye and the optical components and how they combine to yield the image rotation that appears in the raw video output of the present invention; the coordinate system of the right eye is seen to be rotated as light travels from the eye to the camera's lens.

FIG. 6 is a frontal illustration of two removable optical filter shields that attach to the remainder of the apparatus of FIG. 1 to obscure certain wavelengths of light while transmitting others.

FIG. 7 is a quasi-frontal, perspective illustration of an alternate embodiment of the present invention as it is being worn by a test subject, and in which the component for imaging the eyes is the camera of a smartphone (or similar self-contained and self-powered image acquisition, analysis, display and reporting device) that is affixed to a base frame which includes a light-occluding cowl that is configured to hold the smartphone directly in front of a test subject's eyes such that its camera is facing the test subject's eyes and its display screen is facing outward so that the subject's eyes can be seen by a clinician who is evaluating the test subject.

### DETAILED DESCRIPTION OF THE PREFERRED EMBODIMENT

Before explaining at least one embodiment of the present invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The present invention 1 is a one- or single-camera, binocular VOG (1CBVOG) system that facilitates high-speed, precisely synchronous VOG recording from both eyes with a single camera and its communication channel to a suitably programmed processing unit, while achieving high-speed, accurate, real-time video analysis by aligning each eye's mediolateral/horizontal and superoinferior/vertical axes with the rows and columns of the camera's image-sensing pixel elements. This 1CBVOG system can measure the movement of both of the eyes of a test subject while the head of the test subject is undergoing a period of prescribed motion or alternative means of vestibular or oculomotor stimulation, such as head reorientation in a gravitational field, Valsalva or external ear pressure application maneuvers, optokinetic stimulation, presentation of visible targets, presentation of sound or head-tapping stimuli, caloric stimulation, electrical stimulation of the vestibular nerve, or other stimuli—wherein, hereinafter, we refer to all these methods of stimulation as simply vestibular or oculomotor stimulation or stimulation. Thus, the present invention is an especially useful device for improving the diagnosis of vestibular, oculomotor, neurologic, perceptual and attentional disorders.

To properly interpret the raw images yielded by the present invention, an image-rotation-correction is necessary to compensate for the image rotation that occurs when a single camera mounted above the eyes in the midsagittal plane images both eyes via a series of reflecting mirrors from effective vantage points that are anterior and inferior of the center of each eye. This is achieved by orienting and aligning the series of mirrors such that a splitting mirror divides the camera's view into left- and right-eye portions and redirects the camera's views laterally; hot mirrors (i.e., mirrors that transmit visible light but reflect infrared light) redirect the camera's lines of sight to view the eyes from vantage points anterior of each eye, and a third s mirror is included to shift the angle of light incident on the camera such that each eye's horizontal axis maps to a vertical column of light sensors in the camera's image sensor. The image-rotation optical element of the present invention provides the advantage of minimizing the computational load that the system's processing unit would otherwise have to overcome in a single-camera system that did not incorporate this element.

Moreover, previously described VOG systems employ only planar mirrors, whereas the present invention optionally incorporates nonplanar (convex or concave) mirrors that partially or completely obviate the need for relatively large and massive lenses; thus, the present invention achieves reductions of system size, mass and moment of inertia and increases in image fidelity and resolution.

Additionally, the present invention can provide a portable, integral display unit mounted on a test subject's face in the position most familiar to clinicians who are most comfortable with direct examination of a test subject's eyes via Frenzel lenses. This is an improvement to the prior art systems that require the clinician to purchase, power, connect, transport and view a separate video display monitor typically mounted on a nearby wall, table or cart, thereby increasing system size and cost and reducing such a system's portability.

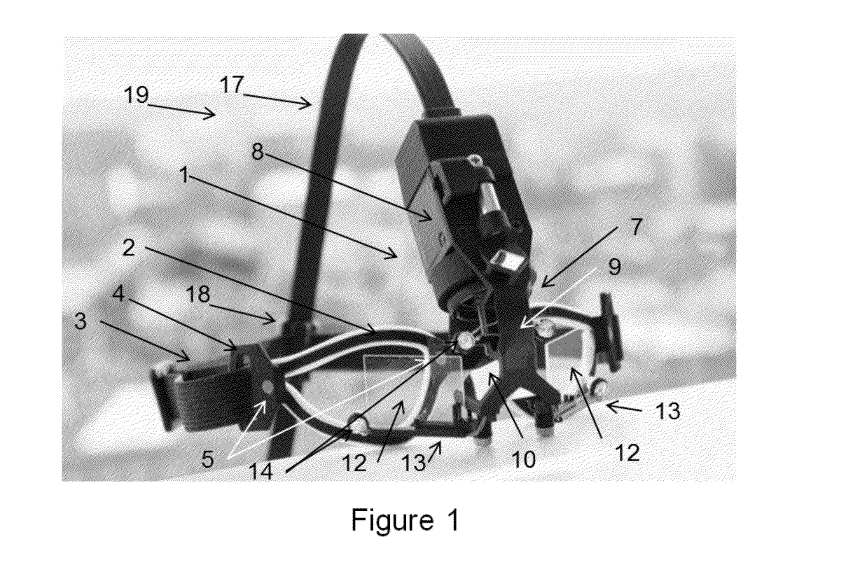

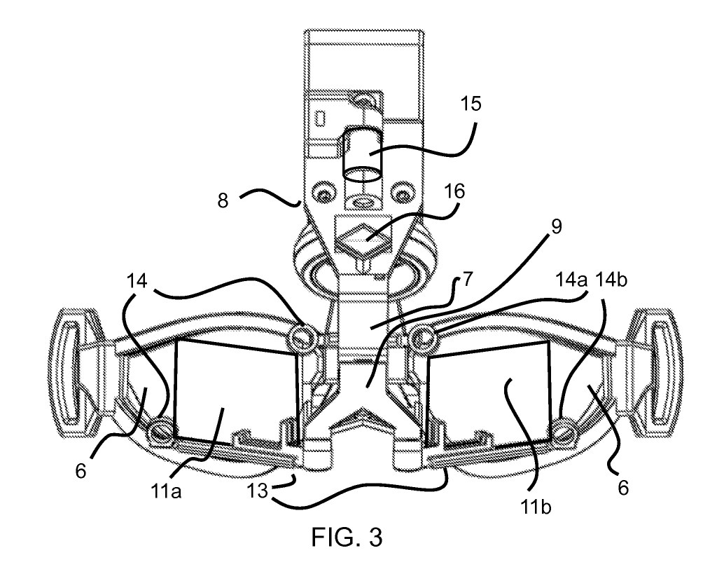

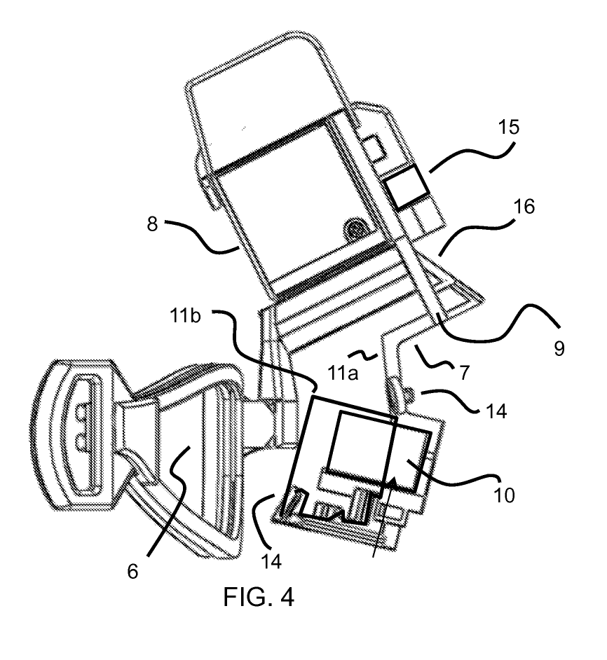

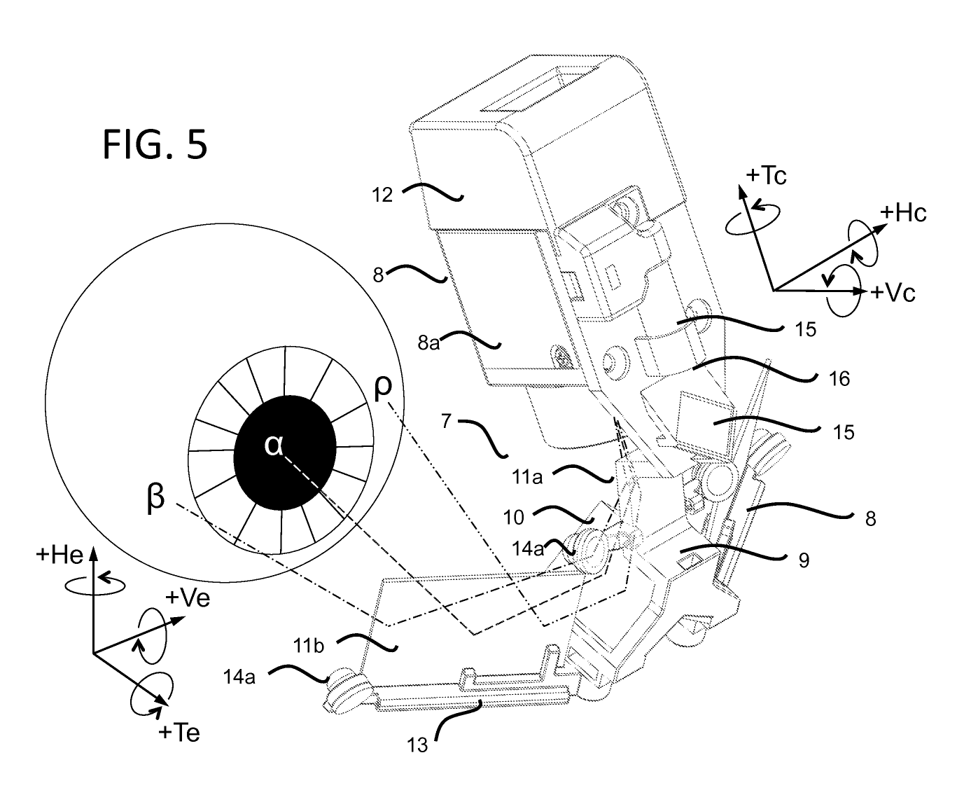

FIGS. 1-5 show an apparatus, device or system for measuring, analyzing, recording and reporting eye movements in a patient according to an embodiment of the present invention. It includes a base frame 2 that is adapted to be worn on the head of a test subject. This base frame is lightweight but strong, and may be formed using 3D printed plastic, plastic formed by other means, metal, carbon fiber or another light and mechanically strong material. It 2 includes: an elastic headband 3 and slots 4 via which the elastic headband couples securely but adjustably with the remainder of base frame 2, magnets 5 for the attachment of optical filter shields 6.

An optical component 7 and binocular optical imaging component 8 are coupled to each other and then coupled to the base frame 2. This optical component 7 includes a superstructure 9, typically made of plastic but alternately made of another suitably strong and light material, which holds a beam-splitting prism mirror 10 and alignment or beam-redirecting IR-reflective (“hot”) mirrors 11a, 11b. This superstructure also supports mirror orientation adjusting frames 13, which allow the user to reorient the mirrors to center the test subject's eye in the portion of the image sensor allotted to each eye.

In one non-limiting, preferred embodiment, the beam-splitting mirror 10 is a single 45 degree right angle prism covered by a reflective gold optical coating on two surfaces that are oriented 90 degrees from each other and 45 degrees from the midsagittal plane. Another coating, such as silver, aluminum, or any other dielectric or reflective film could be used in place of gold, depending on the particular wavelengths of illumination employed in a particular realization of the invention. The prism is positioned symmetrically about the test subject's midsagittal plane close to the test subject's nose and oriented so that a single image frame sampled by the binocular imaging component's camera 8a via the beam-splitting mirror and alignment or beam-redirecting mirrors 11a, 11b includes images of both eyes imaged at approximately equal working distance and angle of incidence. In this embodiment, the beam-redirecting mirror is a single mirror positioned between the camera's lens and the splitter mirror and oriented so that a vector perpendicular to its surface lies in the test subject's midsagittal plane and is within a prescribed number of degrees from the camera's optic axis.

In this embodiment, each of two alignment or beam-redirecting mirrors is a single mirror positioned between the splitter mirror and one eye and oriented so that the image of that eye conveyed to the camera appears as though the camera were viewing that eye from a vantage point below the center of the eye and along an optical axis vector approximately in a plane parallel to the test subject's midsagittal plane but passing through the center of the eye and pupil when the eye is oriented such that its optic axis approximately aligns with the camera's optic axis.

In an alternative, non-limiting preferred embodiment, the beam-splitting mirror is an isosceles triangle prism covered by a reflective optical coating on two surfaces that are 2Θ degrees from each other and Θ degrees from the midsagittal plane, where Θ is any real number between 0 and 90 degrees. The prism and alignment or beam- redirecting mirrors are positioned symmetrically about the test subject's midsagittal plane so that a single image frame sampled by camera 8a via the splitting mirror and beam-redirecting mirrors includes images of both eyes imaged at approximately equal working distance and angle of incidence. Varying Θ to values other than 45 allows one to adjust the effective working distance and magnification of the optical system and camera. The planar mirror surfaces may optionally be replaced by curved mirror surfaces, which reduce or obviate the need for additional lenses in the optical component and may therefore reduce the mass and production cost of the device.

An illumination source 14 of LEDs, which may emit IR or visible light, is housed in the optical component and optionally in the base frame.

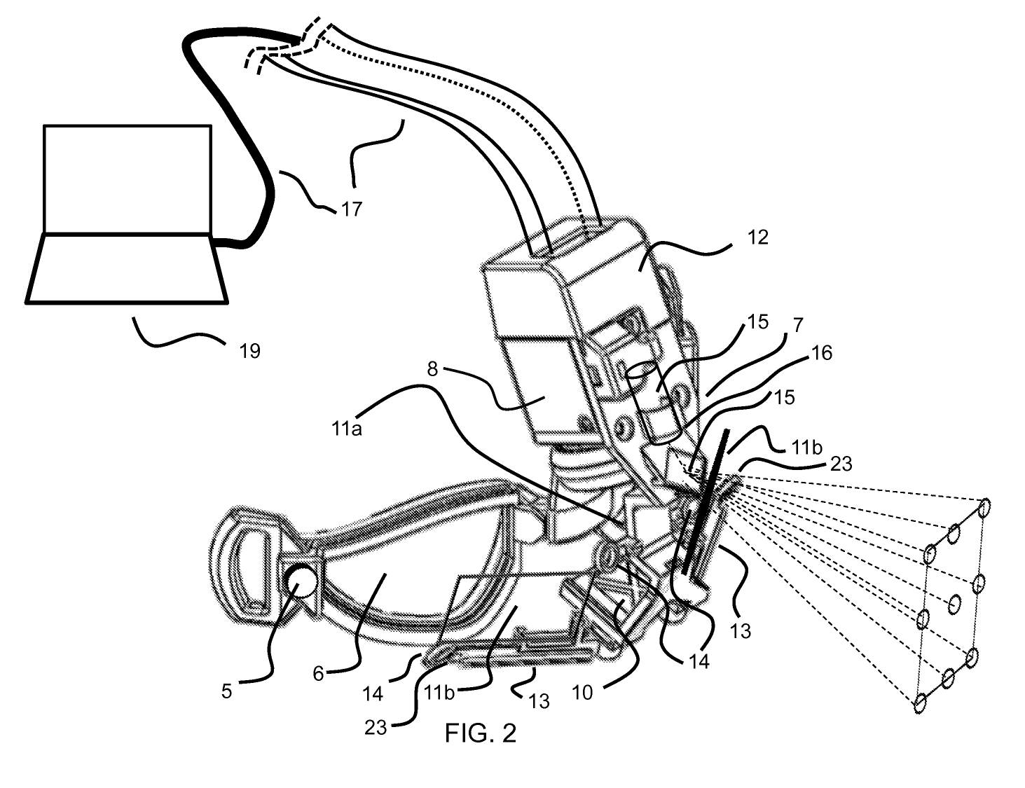

A means for projecting a laser line or a calibration laser 15 and diffraction grating 16 is coupled to the optical component's superstructure to facilitate calibration of this 1 CBVOG system. This diffraction grating is generally affixed to the laser and oriented so that the laser projects a rectangular grid of a plurality (typically 5 or 9) illuminated points on a surface that is perpendicular to the test subject's naso-occipital axis and located in front of the subject so that the illuminated points can serve as visual targets at know positions during calibration. See FIG. 2. Alternatively, the diffraction grating may be configured so that a test subject can rotate it to adjust the orientation of the projected laser line until the test subject perceives the projected laser line as being either vertical or horizontal with respect to the earth, thereby enabling measurement of the subjective visual vertical and/or subjective visual horizontal. The projected laser line is preferably a green or red laser line.

A communication interface 17 consisting of a flat USB3 cable is part of the binocular optical imaging component 8 and communicates the sequence of the digital image outputs of this component's camera 8a to a computing device 19 having a processing unit or processor, a display monitor and a storage device that is programmed to use this sequence of digital images to quantify and measure the movement of both of the eyes of a test subject.

In the preferred embodiment, the system's camera 8a is a high speed USB3 digital camera with a global shutter to ensure synchronous image acquisition of both eyes; however, alternate cameras using alternate communication protocols and their corresponding cables or wireless interfaces 17 could be substituted.

Additionally, the system's computing device 19 is programmed to account for and correct for the possibility that the axis of eye rotation for horizontal (yaw) motion and the axis of eye rotation for vertical (pitch) components of eye rotation fail to intersect, thereby reducing eye movement measurement errors.

The present invention may also include a motion or inertial sensor 12 that is adapted to output a data signal that is a measure of the movement of the base frame and head orientation of a test subject. Such a sensor may be configured to be immobilized with respect to the head by being: (a) held between the teeth of a test subject, or (b) inserted into the test subject's pinna and ear canal. A combination of such sensors may also be adapted to monitor the angular velocity of a test subject's head movements about each of three mutually orthogonal axes of rotation. These axes may be approximately aligned with the mean anatomic axes of the inner ear labyrinths' semicircular canals.

The data signal from this inertial sensor 12 is used to measure the orientation and angular velocity of the head, against which simultaneously measured eye orientation and angular velocity are compared to evaluate the performance of vestibular and oculomotor reflexes.

The entire optical 7 and imagining 8 components of the present invention may be detached from the base frame, to allow a clinician to readily select a different base frame of a shape and size most comfortable for the test subject. Each base frame 2 has curved slots within which a restraining elastic band used to secure the base frame to the test subject's head can be readily slid to adjust the pitch orientation of the base frame with respect to the test subject's facial anatomy. The curved slots have ridges that grasp the elastic band when it is tightened, preventing relative motion between the band and base frame during head movement. The segment of the apparatus holding the alignment or beam-redirecting IR-reflective mirrors allows the user to adjust their orientation and then lock them in that new orientation while a test subject is wearing the apparatus.

The illumination source 14 of the present invention may include at least one visible light LED 14a that emits visible light with sufficient intensity to cause pupil constriction to a diameter smaller than occurring under IR lighting alone, and IR LEDs 14b that provide illumination for video-oculographic imaging. This visible light emitting LED therefore facilitate analysis of eye movements, by reducing the likelihood of a large, dilated pupil being partially obscured by the eyelid. This visible light can also augment or supplant the action of optical filter shields by effectively preventing the eyes from seeing and fixating otherwise-visible items such as Earth-stationary wall edges or head-fixed goggle edges, which might otherwise cause measurement artifacts through visual enhancement or suppression, respectively, of the vestibulo-ocular reflex.

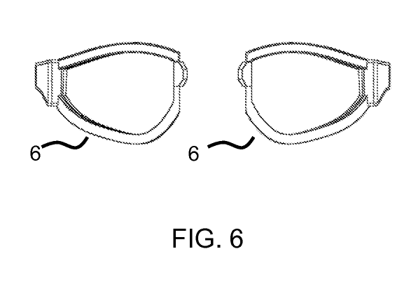

FIG. 6 shows further details of the present invention's optical filter shield 6. The optical filters within the shields may be stackable and may each be single layer or multilayer filters, the latter arrangement allowing passage of more than one discrete spectral band of light, such as a narrow band centered on the emission band of a helium-neon red laser or a green (for example, 532 nm) or other color laser, in addition to transmitting IR light, while blocking other visible light. Each shield has an outer rim that is held to the base frame by magnets or by hook and loop fasteners. Such filter shields may be made of high transmission, low distortion, light-weight, flexible plastic, which is relatively inexpensive, or optically-coated glass, which is relatively more massive and expensive but more rugged.

Alternatively, these shields may be thought of as: (a) a first set of long-pass optical filter shields that reversibly cover each eye and allow infrared light to pass while blocking light of any wavelength visible to humans, (b) a second set of detachable, band-pass optical filter shields that allow transmission of visible light over a narrow range centered on the peak emission wavelength of either a red or green or other color laser—such filters allow the test subject to see a calibration pattern projected using red or green or other color light, respectively, on a screen of wall in an examination room, while effectively remaining in darkness when the exam room is illuminated by light that does not include sufficiently intense spectral components in the pass-band range of the filters, and (c) a third set of detachable optical filter shields that include a stack of three filters, including a short-pass, a band-stop, and a long-pass filter, the combination of which results in a dual pass-band filter that allows transmission of infra-red light and also allows transmission of visible light over a narrow range centered on the peak emission wavelength of either a red or green or other color laser.

The present invention may also include a green or red or other color laser line projected by a laser through a diffraction grating 16 on a surface perpendicular to the test subject's naso-occipital axis, wherein the test subject can rotate the diffraction grating to adjust the orientation of the line until he perceives the line as being vertical or horizontal, thereby providing a measure of subjective visual vertical and subjective visual horizontal, which may be abnormal in test subjects with abnormalities of inner ear, oculomotor, visual or neurologic function. Rotation of the diffraction grating may be accomplished either via direct manual rotation or via control of an electrical or magnetic motor.

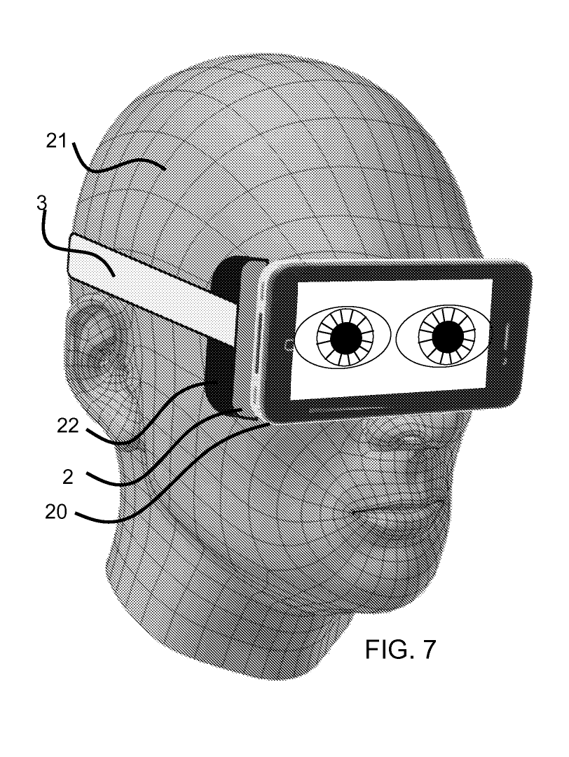

FIG. 7 shows an alternative, non-limiting, preferred embodiment of the invention, wherein the imaging component 8 is a consumer-grade smartphone 20 that has a front and a rear side or face, and wherein the smartphone's main display is in the front face and faces outward and its video cameras is in the rear face and faces the test subject, with the optical component adapted such that the smartphone's video camera can image one or both eyes simultaneously at an effective working distance and depth of field that allows the system to obtain and display sharp images of one or both eyes in test subjects with varying head size and interpupillary distance. In this embodiment, the base frame 2 is adapted to hold the smartphone 20 and immobilize it with respect to the test subject's head in a position that minimizes inertial torques and forces on the smartphone when the subject's head moves. The smartphone has the ability to transfer data and images wirelessly to other computers and/or the internet, thereby allowing for real-time remote viewing of eye images and data and for post-hoc analysis on a remote computer. Wherein, for the purpose of this disclosure, the term smartphone denotes not only devices that can be used for telephony (examples of which include, but are not limited to, the Apple iPhone, Samsung Galaxy, HTC One and LG G series phones) but also similarly capable and sized device with image acquisition, analysis and display capability with a similar form factor, camera, inertial sensor, display, central processing unit (such as, but not limited to, the Apple iTouch).

The base frame has an outer edge that includes a light-occluding cowl 22 that prevents visible light from entering the region imaged by the camera. IR LEDs 14 incorporated into the base frame and located in the region imaged by the camera (i.e., the volume of space defined by the boundaries of the rear side of the smart phone, the light-occluding cowl and the test subject's face—the confines of the light-occluding cowl) provide illumination for video-oculographic imaging. Visible light LEDs incorporated into this same volume of space may optionally illuminate the eyes to constrict the pupils, as would be useful in a subject who cannot focus on a nearby object sufficiently to partly or completely suppress the vestibulo-ocular reflex. In this embodiment, the optical system is adapted to also fit within this volume of space and to image one or both eyes while displaying the image sequence video on the smartphone's screen to a clinician examiner and optionally recording the image sequence video in the smartphone's memory.

The optical system for this embodiment is complicated by the fact that the camera's optic axis is not located at the center of the smartphone's rear face and therefore not in the midsagittal plane of the test subject; it is instead located near the perimeter of the smartphone's rear face. To facilitate imaging both eyes despite the camera's lens being a different distance from each, a combination of prisms, planar and curved mirrors, lenses, graded index of refraction (GRIN) lenses, and optical conduits are embedded within the frame and situated so as to convey a magnified image of each eye to one half of the camera's image sensor, wherein that magnified image of each eye is acquired from an effective vantage point that is within 60 degrees of the eye's optic axis when at rest in neutral primary position.

The present invention should also be recognized as a method for measuring, quantifying and reporting vestibular, oculomotor, neurologic, perceptual and visual function of a test subject. In this method:
(a) the 1CBVOG system disclosed herein is secured to the test subject's head;
(b) a light source projects a calibration pattern grid on a surface approximately perpendicular to the test subject's naso-occipital axis;
(c) the test subject is instructed to visually fixate each point on the calibration pattern grid while the 1CBVOG system measures the angular positions of the test subject's eyes in 1, 2 or 3 dimensions;
(d) the test subject is then instructed to make saccadic eye movements between calibration pattern grid points while the 1CBVOG system measures the eye movements and reports them as a measure of saccadic function;
(e) the test subject is next instructed to smoothly follow a moving visual target while the system described above measures the eye movements and reports them as a measure of smooth pursuit function;
(f) the test subject is then instructed to watch an optical flow pattern on a screen or other visual large-field visual display while the 1CBVOG system measures the eye movements and reports them as a measure of optokinetic response function;
(g) the test subject's head is rotated about axes approximately parallel to the mean axes of the inner ear semicircular canals as the test subject views a distant Earth-fixed target while the 1CBVOG system measures the eye movements and reports them as a measure of visually-enhanced vestibulo-ocular reflex function;
(h) the test subject's head is rotated about axes approximately parallel to the mean axes of the inner ear semicircular canals with the test subject in darkness, while the 1CBVOG system measures the eye movements and reports them as a measure of vestibulo-ocular reflex function in the absence of visual cues;
(i) the test subject is instructed to manipulate a controller as required to orient a laser line projected on a surface in front of him until it is, in his estimation, Earth-vertical, with the laser line initially being set at a random orientation prior to each trial, the true angle of the laser line at the completion of each test trial being a measure of the subject's subjective visual vertical function;
(j) the test subject is instructed to manipulate a controller as required to orient a laser line projected on a surface in front of him until it is, in his estimation, Earth-horizontal, with the laser line initially being set at a random orientation prior to each trial, the true angle of the laser line at the completion of each test trial being a measure of the subject's subjective visual horizontal function; and
(k) a summary report of functional test results is generated and reported to the examining clinician.

Although the foregoing disclosure relates to preferred embodiments of the invention, it is understood that these details have been given for the purposes of clarification only. Various changes and modifications of the invention will be apparent, to one having ordinary skill in the art, without departing from the spirit and scope of the invention.

We claim:

1. A single-camera, binocular, video-oculographic system for measuring the movement of both of the eyes of a test subject while said test subject is undergoing a period of vestibular or oculomotor stimulation, said system comprising:
a base frame adapted to fit onto and be immobilized with respect to the head of said test subject,
a binocular imaging component adapted to capture a sequence of images containing both of the eyes of said test subject during said period of stimulation, wherein said binocular imaging component is attached to said base frame and includes a single video camera having an optic axis that is oriented to lie approximately within the midsagittal plane of said test subject,
an optical component adapted to allow said binocular imaging component to capture said sequence of images containing both of the eyes of said test subject simultaneously and synchronously during said period of stimulation, wherein both said eyes are imaged at the same effective moment in time and from effective vantage points that are within a prescribed number of degrees of the optic axis of each eye when that eye is in the center of its range of motion,
an illumination source adapted to provide illumination during the capture of said sequences of images, and
a computing device configured to communicate with said single camera so as to quantify and measure the movement of both of the eyes of said test subject by utilizing said captured sequence of images.

2. The system as recited in claim 1, wherein:
said optical component includes:
a beam splitting mirror approximately centered on the test subject's midsagittal plane and aligned with the optic axis of said camera, and
a plurality of alignment mirrors that are each aligned with said beam splitting mirror and configured so that said camera simultaneously images both eyes at approximately the same effective working distance and magnification without occluding the central region of the visual field of either of the eyes of said test subject.

3. The system as recited in claim 2, further comprising:
s a pair of detachable optical filter shields, each of which includes an outer rim that encloses an optical filter, and each of which is configured to reversibly cover and occlude vision in one of the eyes of said test subject, and
wherein said optical filter is chosen from the group including: (a) a long-pass optical filter configured to allow infrared light to pass while blocking light of any wavelength visible to humans, (b) a band-pass optical filter that allows transmission of visible light over a narrow range centered on the peak emission wavelength chosen from the group of either a red or green or other color laser, and (c) a stack of three optical filters, including a short-pass, a band-stop, and a long-pass filter, the combination of which results in a dual pass-band filter that allows transmission of infra-red light and also allows transmission of visible light over a narrow wavelength range.

4. The system as recited in claim 2, further comprising:
a motion sensor having a plurality of axes of sensitivity that are adapted to be immobilizably affixed to the head of said test subject so as to approximately align with the mean anatomic axes of the inner ear labyrinths' semicircular canals of said test subject and to output a data signal that is a measure of the orientation and movement of the head of said test subject.

5. The system as recited in claim 3, further comprising:
a diffraction grating oriented perpendicular to the naso-occipital axis of said test subject,
a means for projecting a visible laser line through said diffraction grating, wherein said diffraction grating configured so that said test subject rotates said diffraction grating to adjust the orientation of said projected laser line until said test subject perceives said projected laser line as being in the group of vertical or horizontal lines.

6. The system as recited in claim 4, further comprising:
a diffraction grating oriented perpendicular to the naso-occipital axis of said test subject,
a means for projecting a visible laser line through said diffraction grating,
wherein said diffraction grating configured so that said test subject rotates said diffraction grating to adjust the orientation of said projected laser line until said test subject perceives said projected laser line as being in the group of vertical or horizontal lines.

7. The system as recited in claim 6, wherein:
said illumination source includes: (a) a lamp and optical band-stop filter combination that emits visible light but excludes light at wavelengths within the visible light pass band of said detachable optical filter shields, and (b) a light-emitting diode that emits visible light with sufficient intensity to cause the pupil of said test subject to constrict to a pupil diameter smaller than that which occurs under infra-red lighting alone.

8. The system as recited in claim 7, wherein:
said computing device is programmed to account for and correct for: (i) the lack of a single common center of eye rotation through which pass both the axis of eye rotation for horizontal motion and the axis of eye rotation for vertical components of eye rotation, and (ii) the failure to intersect of the axis of eye rotation for horizontal (yaw) motion and the axis of eye rotation for vertical (pitch) motion.

9. The system as recited in claim 8, wherein:
said optical component includes at least one element chosen from the group including a nonplanar mirror, a mirror having a gold reflective optical coating, a graded index lens, and an optical conduit.

10. The system as recited in claim 2, wherein:
said single video camera is part of a smartphone that is of the type having a front and a rear side, with said single camera being located on said back side and said smartphone further having a display screen located on said front side,
said base frame has outer edges that include a light-occluding cowl and said base frame is adapted to hold said smartphone in a position that orients the optic axis of said video camera to face said test subject,
said beam splitting mirror and plurality of alignment mirrors are further adapted to fit within the confines of said light-occluding cowl and do occlude the central region of the visual field of either of the eyes of said test subject, and
said illumination source adapted to fit within the confines of said light-occluding cowl.

11. A method that utilizes a single-camera, binocular, video-oculographic system for measuring the movement of both of the eyes of a test subject while said test subject is undergoing a period of vestibular or oculomotor stimulation, said method comprising the steps of:
utilizing a base frame adapted to fit onto and be immobilized with respect to the head of said test subject,
utilizing a binocular imaging component adapted to capture a sequence of images containing both of the eyes of said test subject during said period of stimulation, wherein said binocular imaging component is attached to said base frame and includes a single video camera having an optic axis that is oriented to lie approximately within the midsagittal plane of said test subject,
utilizing an optical component adapted to allow said binocular imaging component to capture said sequence of images containing both of the eyes of said test subject simultaneously and synchronously during said period of stimulation, wherein both said eyes are imaged at the same effective moment in time and from effective vantage points that are within a prescribed number of degrees of the optic axis of each eye when that eye is in the center of its range of motion,
utilizing an illumination source adapted to provide illumination during the capture of said sequences of images, and
utilizing a computing device configured to communicate with said single camera so as to quantify and measure the movement of both of the eyes of said test subject by utilizing said captured sequence of images.

12. The method as recited in claim 11, wherein:
said optical component includes:
a beam splitting mirror approximately centered on the test subject's midsagittal plane and aligned with the optic axis of said camera, and
a plurality of alignment mirrors that are each aligned with said beam splitting mirror and configured so that said camera simultaneously images both eyes at approximately the same effective working distance and magnification without occluding the central region of the visual field of either of the eyes of said test subject.

13. The method as recited in claim 12, further comprising the step of:
utilizing a pair of detachable optical filter shields, each of which includes an outer rim that encloses an optical filter, and each of which is configured to reversibly cover and occlude vision in one of the eyes of said test subject, and
wherein said optical filter is chosen from the group including: (a) a long-pass optical filter configured to allow infrared light to pass while blocking light of any wavelength visible to humans, (b) a band-pass optical filter that allows transmission of visible light over a narrow range centered on the peak emission wavelength chosen from the group of either a red or green or other color laser, and (c) a stack of three optical filters, including a short-pass, a band-stop, and a long-pass filter, the combination of which results in a dual pass-band filter that allows transmission of infra-red light and also allows transmission of visible light over a narrow wavelength range.

14. The method as recited in claim 12, further comprising the step of:
utilizing a motion sensor having a plurality of axes of sensitivity that are adapted to be immobilizably affixed to the head of said test subject so as to approximately align with the mean anatomic axes of the inner ear labyrinths' semicircular canals of said test subject and to output a data signal that is a measure of the orientation and movement of the head of said test subject.

15. The method as recited in claim 13, further comprising the step of:
utilizing a diffraction grating oriented perpendicular to the naso-occipital axis of said test subject,
utilizing a means for projecting a visible laser line through said diffraction grating,
wherein said diffraction grating configured so that said test subject rotates said diffraction grating to adjust the orientation of said projected laser line until said test subject perceives said projected laser line as being in the group of vertical or horizontal lines.

16. The method as recited in claim 14, further comprising the step of:
utilizing a diffraction grating oriented perpendicular to the naso-occipital axis of said test subject,
utilizing a means for projecting a visible laser line through said diffraction grating,
wherein said diffraction grating configured so that said test subject rotates said diffraction grating to adjust the orientation of said projected laser line until said test subject perceives said projected laser line as being in the group of vertical or is horizontal lines.

17. The method as recited in claim 16, wherein:
said illumination source includes: (a) a lamp and optical band-stop filter combination that emits visible light but excludes light at wavelengths within the visible light pass band of said detachable optical filter shields, and (b) a light-emitting diode that emits visible light with sufficient intensity to cause the pupil of said test subject to constrict to a pupil diameter smaller than that which occurs under infra-red lighting alone.

18. The method as recited in claim 17, wherein:
said computing device is programmed to account for and correct for: (i) the lack of a single common center of eye rotation through which pass both the axis of eye rotation for horizontal motion and the axis of eye rotation for vertical components of eye rotation, and (ii) the failure to intersect of the axis of eye rotation for horizontal (yaw) motion and the axis of eye rotation for vertical (pitch) motion.

19. The method as recited in claim 18, wherein:
said optical component includes at least one element chosen from the group including a nonplanar mirror, a mirror having a gold reflective optical coating, a graded index lens, and an optical conduit.

20. The method as recited in claim 12, wherein:
said single video camera is part of a smartphone that is of the type having a front and a rear side, with said single camera being located on said back side and said smartphone further having a display screen located on said front side,
said base frame has outer edges that include a light-occluding cowl and said base frame is adapted to hold said smartphone in a position that orients the optic axis of said video camera to face said test subject,
said beam splitting mirror and plurality of alignment mirrors are further adapted to fit within the confines of said light-occluding cowl, and
said illumination source adapted to fit within the confines of said light-occluding cowl.

21. The method as recited in claim 14, wherein:
said single video camera is part of a smartphone that is of the type having a front and a rear side, with said single camera being located on said back side and said smartphone further having a display screen located on said front side,
said base frame has outer edges that include a light-occluding cowl and said base frame is adapted to hold said smartphone in a position that orients the optic axis of said video camera to face said test subject,
said beam splitting mirror and plurality of alignment mirrors are further adapted to fit within the confines of said light-occluding cowl, and said illumination source adapted to fit within the confines of said light-occluding cowl.

22. The method as recited in claim 11, further comprising the step of:
causing said test subject to smoothly follow a moving visual target while said system measures the eye movements of said test subject to assess said test subject's smooth pursuit function,
causing said test subject to watch an optical flow pattern on a visual display while said system measures the eye movements of said test subject to assess said test subject's optokinetic response function, and
causing said test subject to perform quick, voluntary eye redirection movements to fixate a series of targets while said system measures the eye movements of said test subject to assess said test subject's saccadic function.

23. The method as recited in claim 11, further comprising the step of:
rotating the head of said test subject about axes approximately parallel to the mean axes of the inner ear semicircular canals as the test subject views a distant Earth-fixed target while said system measures the eye movements of said test subject to assess the visually-enhanced, vestibulo-ocular reflex function of said test subject, and
rotating the head of said test subject about axes approximately parallel to the mean axes of the inner ear semicircular canals with said test subject in darkness, while said system measures the eye movements of said test subject to assess the vestibulo-ocular reflex function in the absence of visual cues of said test subject.

24. The method as recited in claim 12, further comprising the step of:
rotating the head of said test subject about axes approximately parallel to the mean axes of the inner ear semicircular canals as the test subject views a distant Earth-fixed target while said system measures the eye movements of said test subject to assess the visually-enhanced, vestibulo-ocular reflex function of said test subject, and
rotating the head of said test subject about axes approximately parallel to the mean axes of the inner ear semicircular canals with said test subject in darkness, while said system measures the eye movements of said test subject to assess the vestibulo-ocular reflex function in the absence of visual cues of said test subject.

25. The method as recited in claim 11, further comprising the step of:
when said system is further configured to project a calibration pattern grid on a surface perpendicular to the test subject's naso-occipital axis,
causing said test subject to visually fixate on each point on said calibration pattern grid while said system measures the angular positions of the test subject's eyes.

26. The method as recited in claim 14, further comprising the step of:
when said system is further configured to project a calibration pattern grid on a surface perpendicular to the test subject's naso-occipital axis,
causing said test subject to visually fixate on each point on said calibration pattern grid while said system measures the angular positions of the test subject's eyes.

27. The method as recited in claim 15, further comprising the step of:
causing said test subject to make saccadic eye movements between said calibration pattern grid points while said system measures the eye movements of said test subject to assess said test subject's saccadic function.

28. The method as recited in claim 16, further comprising the step of:
causing said test subject to make saccadic eye movements between said calibration pattern grid points while said system measures the eye movements of said test subject to assess said test subject's saccadic function.

29. The method as recited in claim 11, further comprising the step of:
when said system further comprises:
a diffraction grating oriented perpendicular to the naso-occipital axis of said test subject,
a means for projecting a visible laser line through said diffraction grating,
wherein said diffraction grating configured so that said test subject rotates said diffraction grating to adjust the orientation of said projected laser line until said test subject perceives said projected laser line as being in the group of vertical or horizontal lines.
causing said test subject to manipulate said means for projecting a visible laser line as required to orient said projected laser line on a surface in front of said test subject until said projected laser line is Earth-vertical, with said projected laser line initially being set at a random orientation prior to each test trial, the true angle of the projected laser line at the completion of each test trial being a measure of the subject's subjective visual vertical function, and
causing said test subject to manipulate said means for projecting a laser line as required to orient said projected laser line on a surface in front of said test subject until said projected laser line is horizontal, with said projected laser line initially being set at a random orientation prior to each test trial, the true angle of the projected laser line at the completion of each test trial being a measure of the subject's subjective visual horizontal function.

30. The method as recited in claim 18, further comprising the step of:
when said system further comprises:
a diffraction grating oriented perpendicular to the naso-occipital axis of said test subject,
a means for projecting a visible laser line through said diffraction grating,
wherein said diffraction grating configured so that said test subject rotates said diffraction grating to adjust the orientation of said projected laser line until said test subject perceives said projected laser line as being in the group of vertical or horizontal lines.
causing said test subject to manipulate said means for projecting a visible laser line as required to orient said projected laser line on a surface in front of said test subject until said projected laser line is Earth-vertical, with said projected laser line initially being set at a random orientation prior to each test trial, the true angle of the projected laser line at the completion of each test trial being a measure of the subject's subjective visual vertical function, and
causing said test subject to manipulate said means for projecting a laser line as required to orient said projected laser line on a surface in front of said test subject until said projected laser line is horizontal, with said projected laser line initially being set at a random orientation prior to each test trial, the true angle of the projected laser line at the completion of each test trial being a measure of the subject's subjective visual horizontal function.
